(19) United States
(12) Plant Patent Application Publication
(10) Pub. No.: US 2008/0184402 P1
(43) Pub. Date: Jul. 31, 2008

(54) Apple tree named 'Lady Laura'

(76) Inventors: Max Davidson, Orange (AU); Grahame Davidson, Orange (AU)

Correspondence Address: KLARQUIST SPARKMAN, LLP, 121 SW SALMON STREET, SUITE 1600, PORTLAND, OR, 97204, US

(21) Appl. No.: 11/493,500

(22) Filed: Jul. 25, 2006

(51) Int. Cl. A01H 5/00

(52) U.S. Cl. PLT/161

(57) ABSTRACT

A new apple variety distinguished by the amount and intensity of over-color of the fruit and the earlier coloration of the fruit.

### CROSS REFERENCE TO RELATED APPLICATIONS

None

### STATEMENT REGARDING FEDERALLY SPONSORED RESEARCH OR DEVELOPMENT

None

### LATIN NAME OF THE GENUS AND SPECIES OF THE PLANT CLAIMED

Malus domestica

### VARIETY DENOMINATION

‘Lady Laura’

### BACKGROUND OF THE INVENTION

The present invention relates to a new and distinct variety of apple tree named ‘Lady Laura.’ The new tree resulted from a spontaneous limb sport mutation of ‘Cripps Pink’ (U.S. Plant Pat. No. 7,880). The ‘Cripps Pink’ tree containing the limb sport of the new variety was discovered growing in a cultivated area in Borenore NSW, Australia.

### BRIEF SUMMARY OF THE INVENTION

The ‘Lady Laura’ variety is distinguished from other apple varieties due to the following unique combination of characteristics: the amount and intensity of over color of the fruit, and the earlier coloration of fruit in comparison to ‘Cripps Pink’.

Asexual reproduction of this new variety by budding and grafting shows that the foregoing characteristics come true to form, are firmly fixed, and are established and transmitted through succeeding propagations.

Certain characteristics of this variety, such as growth and color, may change with changing environmental conditions (such as light, temperature, moisture, nutrient availability, or other factors). Color descriptions and other terminology are used in accordance with their ordinary dictionary descriptions, unless the context clearly indicates otherwise. Color designations are made with reference to The Royal Horticultural Society (RHS) Colour Chart.

### BRIEF DESCRIPTION OF THE DRAWING

FIG. 1 is a photograph showing typical fruit of the new variety.

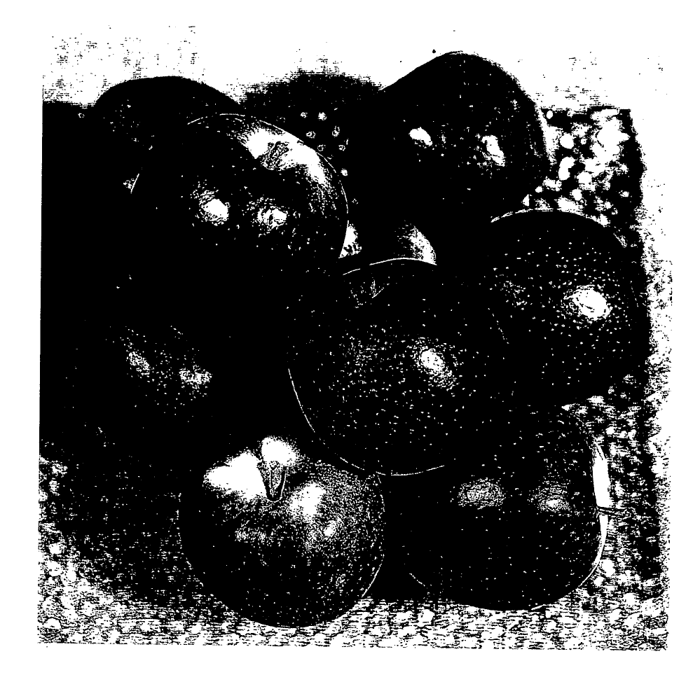

The accompanying color photograph shows typical specimens of the fruit of this new apple tree variety and depict the color as nearly true as is reasonably possible to make the same in a color illustration of this character. It should be noted that colors may vary, for example due to lighting conditions at the time the photograph is taken. Therefore, color characteristics of this new variety should be determined with reference to the observations described herein, rather than from the photograph alone.

### DETAILED DESCRIPTION

### BOTANICAL

The following detailed description of the ‘Lady Laura’ variety is based on observations of the original limb sport and asexually reproduced progeny. The observed progeny are trees which were four years of age and growing on Exemla 9 rootstock (unpatented) in Taggerty, Victoria, Australia.
Scientific name: Malus domestica ‘Lady Laura’
Parentage: Spontaneous limb sport mutation of ‘Cripps Pink’.
Tree:
   Vigor.—Medium.
   Overall shape.—Upright.
   Height.—About 5 to 6 feet.
   Width.—Overall spread of about 4 feet.
   Caliper.—Approximately 3 inches.
   Trunk.—Medium stocky.
   Trunk bark texture.—Medium rough with lenticels.
   Trunk bark color.—Grey/Green/Cream (RHS 195B).
   Patches or other markings.—Lenticels one-fourth to one-half inch long.
   Primary branches.—Upright.
   Branch color.—One-year old branches are green (RHS 144B) in color, while two-year old branches are green-brown (RHS 199B) in color.
   Branch pubescence.—Present.
   Branch lenticels.—Medium density, approximately 38 per square inch; greyed-orange (RHS 164D) in color.
   Internodes.—Average internode length is about one and one-sixteenth inch on a one-year old shoot.
   Bearing.—Annual. No particular disease resistance or susceptibility observed.
Leaves:
   Texture.—Medium thick.
   Sheen.—Medium dull.
   Length.—About 3½ inches to about 5 inches, averaging about 4¼ inches.
   Width.—About 2¼ inches to about 2¾ inches, averaging about 2½ inches.
   Petiole.—About 1 and one-eighth inches long; red-green in color (red RHS 179A and green 148D); about one-sixteenth inch in diameter.
   Margin.—Irregularly serrated.
   Tip shape.—Acutely pointed.
   Leaf color.—Upper leaf surface: Green (RHS 137A). Lower leaf surface: Light green (RHS 147B). Vein: Light green (RHS 147C).
   Pubescence.—yes. The length, width, thickness and other measurements were obtained from observations of ten typical leaves in late spring.
Flowers:
   Size.—Medium size, typical flower measuring about 30 mm across.
   Color.—Unopened bud: Pink (RHS 67B). Opened flower: White to very pale pink (white RHS 155D and pink RHS 65D).
   Petals.—5 petals per flower; round to ovate in shape; about five-eighths (to slightly larger) inch long.
   Stamen.—16 stamens, each about 9 mm long and cream white (RHS 155D) in color.
   Anthers.—Pale yellow (RHS 6D) in color.
   Pistil.—Stigma is about 8 mm long; 5 styles, fused at base, and pale brown (RHS 162D) in color.
   Sepals.—About 6 mm and about 3 mm wide; recurved shape; light green (RHS 142D) in color; pubescence present.
   Fragrance.—Light.
   Bloom season.—Early to mid-full bloom observed. Timing varies depending on chill units.
Fruit: (Observations from a limited number of typical fruit in April 2005)
   Size.—About 2 and seven-eighths inches long and 3 inches wide.
   Form.—Round oblong.
   Cavity.—Medium broad to deep.
   Basin.—About seven-sixteenths inch deep and about seven-eighths inch wide; pubescence present.
   Stem.—About five-eighths inch long and one-eighth inch in diameter; yellow green (RHS 152D) in color.
   Locules.—Mostly closed locules.
   Skin.—Medium thick.
   Lenticels.—Prominent.
   Color.—General color effect: Solid dark pink-red (RHS 47A-50A) over yellowish ground color (RHS 10B). Ground color: Green to yellow (RHS 10B). Overcolor: Dark pink to red (RHS 47A to 50A). Overcolor percentage of approximately 90% compared to ‘Cripps Pink’ of approximately 30-80%. The overcolor is more intense and there is a greater percentage of color and more even overcolor that ‘Cripps Pink.’ The fruit achieve overcolor approximately 14 days earlier than ‘Cripps Pink.’ Russetting: Absent.
   Fruit properties at maturity (based on 10 apples tested in April 2005).—Firmness: About 8 kg, averaging about 8.5 kg/cm². Soluble solids: Averaging about 15%. Flavor: Mild. Juiciness: Medium. Flesh color: Creamy white (RHS 4D). Aroma: Mild pleasant.
   Seed.—About 2 seeds per cell; acute shaped; about three-eighths inch long and about three-sixteenth inch wide; brown (RHS 174A) in color.
   Fruit production.—First picking date in Victoria, Australia was about 4^{th} of April, and last picking date was about 10^{th} of April 2005.
   Storage.—Fruit can be stored up to 4 months in cold storage (34° F.).
   Usage.—Eating.

We claim:

1. A new and distinct variety of apple tree, substantially as herein shown and described.
